(19) United States
(12) Patent Application Publication
(10) Pub. No.: US 2020/0048208 A1
(43) Pub. Date: Feb. 13, 2020

(54) TREATMENT OF DISEASES OR DISORDERS CAUSED BY INDUCED NFkB TRANSCRIPTIONAL ACTIVITY

(71) Applicant: Senex Biotechnology, Inc., Columbia, SC (US)

(72) Inventors: Donald C. PORTER, Columbia, SC (US); Igor B. RONINSON, Lexington, SC (US)

(73) Assignee: Senex Biotechnology, Inc., Columbia, SC (US)

(21) Appl. No.: 16/549,794

(22) Filed: Aug. 23, 2019

Related U.S. Application Data
(63) Continuation of application No. 14/343,947, filed on Jun. 24, 2014, now abandoned.
(60) Provisional application No. 61/534,081, filed on Sep. 13, 2011.

(51) Int. Cl. C07D 239/94; A61K 31/517

(52) U.S. Cl. CPC C07D 239/94; A61K 31/517

(57) ABSTRACT

The invention provides a method for treating a disease or disorder in a mammal which is caused by induced NFkB transcriptional activity in cells of the mammal, the method comprising administering to the mammal a compound that specifically inhibits one or more of CDK8 and CDK19.

### BACKGROUND OF THE INVENTION

### Field of the Invention

The invention relates to the treatment of diseases or disorders caused by induced NF-κB transcriptional activity.

### SUMMARY OF THE RELATED ART

The nuclear factor-κB (NFκB) family of transcription factors, comprising dimers of NFκB and Rel family proteins, has been implicated in several major diseases (Gupta et al., 2010; Marcu et al., 2010; Roman-Blas and Jimenez, 2008; O'Sullivan et al., 2007; Sethi et al., 2008; Melisi and Chiao, 2007). NFκB is activated by a variety of signals, including cytokines, such as tumor necrosis factor-TNF-α. (TNF-α) and interleukin 1β (ILβ), chemokines, bacterial and viral products and free radicals. Most of the inducers activate NFκB through the canonical pathway (FIG. 1), which involves phosphorylation of NFκB-binding inhibitory IκB proteins by IκB kinases (IKK), followed by proteasomal degradation of IκB. NFκB dimers released from IκB inhibition enter the nucleus, where they undergo post-translational modifications and bind to specific cis-regulatory sequences in the promoters of NFκB-responsive genes, in association with coactivator proteins (principally p300/CBP protein acetylases) and RNA polymerase II (Pol II) (Hayden and Ghosh, 2008; Roman-Blas and Jimenez, 2008). Certain signals activate NFκB through alternative pathways, mediated by IKK or IκB proteins, such as the non-canonical pathway triggered by lymphotoxin-TNF-α. or RANKL (a cytokine involved in bone resorption and dendritic cell maturation) and regulating a distinct class of genes (Gupta et al., 2010; Hayden and Ghosh, 2008; Roman-Blas and Jimenez, 2008).

NFκB upregulates genes involved in immune inflammatory responses, acute-phase inflammatory responses, oxidative stress responses, cell adhesion and differentiation; NFκB activation has been implicated in inflammatory arthritis and other rheumatic disorders (Roman-Blas and Jimenez, 2008; O'Sullivan et al., 2007). Constitutive NFκB activation also occurs in many cancers and has been linked to tumor cell resistance to apoptosis and necrosis, increased proliferation, angiogenesis and metastasis (Gupta et al., 2010; Melisi and Chiao, 2007; Shen and Tergaonkar, 2009; Richmond, 2002; Sethi et al., 2008). NFκB stimulates gene expression of several human viruses including HIV (Tergaonkar, 2006). Naturally, NFκB has become a major target for drug development (Gupta et al., 2010). Many existing drugs (including non-steroidal anti-inflammatory drugs (NSAIDs) and glucocorticoids) were found to inhibit NFκB, and a number of compounds are undergoing development as NFκB inhibitors, although no drugs aimed specifically at NFκB have yet been approved (Gupta et al., 2010; Tergaonkar, 2006; Sethi et al., 2008; Roman-Blas and Jimenez, 2008). The principal steps of the NFκB pathway targeted by the existing inhibitors (Gupta et al., 2010; Roman-Blas and Jimenez, 2008; Melisi and Chiao, 2007; Sethi et al., 2008) are indicated with stars in FIG. 1. Many of these inhibitors target IKK, and another major class blocks the proteasome activity. Some NFκB inhibitors target NFκB-inducing signals, while others block NFκB translocation from the cytoplasm to the nucleus, inhibit NFκB modifications or DNA binding. NFκB gene expression inhibitors (such as siRNA) are also being developed. The most NFκB-specific class of existing pharmaceutical inhibitors target IKK. However, the first IKK inhibitor to go through cancer clinical trials, CHS-828 (Hassan et al., 2006), showed high toxicity and no objective responses in Phase I (von Heideman et al., 2010). A proteasome inhibitor, Bortezomib, with strong NFκB-inhibitory activity has been approved for the treatment of multiple myeloma (Hideshima et al., 2009). Like other proteasome inhibitors, Bortezomib is cytotoxic, and clinical experience showed substantial toxicity, with Bortezomib-induced peripheral neuropathy observed in 37-44% of patients (Cavaletti and Jakubowiak, 2010). IKK and proteasome inhibitors, which shift the equilibrium between IκB-bound and free NFκB decrease both basal and induced NFκB activity; such inhibitors therefore may interfere with normal physiological functions of NFκB. In contrast, the RANKL inhibitor denosumab that affects only a subset of NFκB-mediated responses (Pageau, 2009) has been approved for bone loss therapy and showed a good safety profile (Hiligsmann and Reginster, 2010).

A stress-specific mechanism of NFκB activation was discovered in the 1990s but has received relatively little attention. This mechanism is the stimulation of NFκB transcriptional activity by p21 (CDKNIA) (Perkins et al., 1997; Poole et al., 2004), a cell cycle inhibitor induced by various types of cellular damage and in the program of senescence (Abbas and Dutta, 2009). p21 binds different cyclin-dependent kinases (CDKs), a family of serine/threonine kinases comprising 21 members in the human genome, which act in a complex with regulatory cyclin proteins. The best-known CDKs (CDK1, 2, 4, 6) are required for transitions between different phases of the cell cycle, but many others function as regulators of transcription or RNA processing (Malumbres et al., 2009). p21 binding usually inhibits CDK activity, but in the case of CDK4, p21 facilitates the assembly of cyclin-CDK complexes and may promote CDK4 activity in vivo (LaBaer et al., 1997). p21 stimulates NFκB activity in reporter assays but does not increase cellular levels of active NFκB (Perkins et al., 1997; Poole et al., 2004). The effect of p21 on NFκB is mediated by the stimulation of p300/CBP coactivator proteins (Perkins et al., 1997; Snowden et al., 2000), and this stimulation is due not to the inhibition of p300/CBP phosphorylation by CDK2 but to an effect on the sumoylation-dependent transcriptional repression domain of p300, CRD1 (Snowden et al., 2000; Gregory et al., 2002; Garcia-Wilson and Perkins, 2005). Studies by one of the instant inventors have demonstrated that p21 expression increases transcription of a large group of genes, many of which have been implicated in cancer, Alzheimer's disease and atherosclerosis; p21 also stimulated all the tested promoters of different viruses (Chang et al., 2000; Chang et al., 2002; Poole et al., 2004). Induction of 5 of 6 tested cellular promoters by p21 was blocked by the IκBα super-repressor, and promoter response to p21 was abrogated by mutating an NFκB element; induction of transcription by p21 was inhibited by Sulindac and some other NSAIDs at concentrations that inhibit NFκB (Poole et al., 2004). Hence, NFκB is a key mediator of the induction of transcription by p21. The transcriptional response to p21 can be mimicked by other CKI proteins (p27 and p16), and therefore it has been termed the CKI pathway.

Two closely related kinases of the CDK family, CDK8 and CDK19 function in the regulation of transcription rather than cell cycle progression (Malumbres et al., 2009). (CDK19 was also called CDC2L6 and CDK11, but the name CDK11 is more often applied to two other proteins). CDK8 and CDK19 (coupled with Cyclin C) are alternative components of a regulatory module of the Mediator complex that connects transcriptional regulators with Pol II (Sato et al., 2004). Little is known about CDK19, which substitutes for CDK8 in the corresponding Mediator modules and may have a different effect from CDK8 in some situations (Tsutsui et al., 2008). On the other hand, CDK8 is known as an oncogene amplified in ˜50% of colon cancers (Firestein and Hahn, 2009), and it has been implicated in pathways involved in stress response. In particular, CDK8 regulates Smad transcriptional activation and turnover in BMP and TGFβ (Alarcon et al., 2009) and acts as a stimulus-specific positive coregulator of p53 target genes (Donner et al., 2007). CDK8 knockdown and knockout studies showed that CDK8 is required for early embryonic development but not needed for the proliferation of any tested cell types (Westerling et al., 2007).

The rationale for NFκB inhibition in the clinic is compelling. However, a new mode of NFκB inhibition that would be geared primarily towards pathological conditions, such as NFκB upregulation in inflammatory arthritis or cancer, is urgently needed.

### BRIEF SUMMARY OF THE INVENTION

The present inventors have discovered compounds (called SNX2-class compounds) that selectively inhibit CDK8/19 and that not only inhibit the induction of NFκB transcriptional activity by p21 but, surprisingly, also prevent the induction of this activity by a canonical NFκB inducer TNF-α, which acts through a well-characterized mechanism unrelated to the CKI pathway. This discovery indicates that SNX2-class compounds and CDK8/19 inhibitors in general have utility in the treatment of a variety of diseases, including but not limited to inflammatory diseases, which are known to be caused by NFκB.

The invention provides a method for treating a disease or disorder in a mammal which is caused by induced NFκB transcriptional activity in cells of the mammal, the method comprising administering to the mammal a compound that specifically inhibits one or more of CDK8 and CDK19. In some embodiments, the induced NFκB transcriptional activity is not induced by the CKI pathway. In some embodiments, the induced NFκB transcriptional activity is induced by the canonical pathway. In some embodiments, the NFκB transcriptional activity has been induced by TNF-α. In some embodiments the induced NFκB transcriptional activity is inhibited without inhibiting the basal NFκB transcriptional activity. In some embodiments, the disease is an inflammatory disease. In some embodiments, the inflammatory bowel disease is Chron's disease or ulcerative colitis. In some embodiments, the compound has a structure selected from the group of structures shown in FIGS. 9A-9B.

### BRIEF DESCRIPTION OF THE DRAWINGS

FIG. 1 shows the canonical pathway for NFκB activation.

FIG. 2 shows dose dependent inhibition by CDK8/19 inhibitor Senexin A of IPTG-induced GFP expression from a NFκB-dependent promoter in HT1080 cells with IPTG-inducible p21 and the structures of CDK8/19 inhibitors SNX2-1-53 (Senexin A) and SNX2-1-139.

FIG. 3 shows the dose dependent effect of CDK8/19 inhibitors SNX2-1-53 (Senexin A) and SNX2-1-139 on normalized GFP expression in untreated and TNFα-treated HT1080-derived reporter cells expressing GFP from a NFκB-dependent promoter.

FIG. 4 shows the effect of Senexin A on the induction of NFkB-regulated genes by TNFa in HEK293 cells, measured using quantitative reverse-transcription PCR (QPCR).

FIG. 5 shows TNFα induction of NFκB-regulated genes in the wild-type and p21−/− HCT116 cells (left) and the effects of Senexin A on the expression of these genes in TNFα-treated cells.

FIG. 6 shows the effects of shRNAs targeting CDK8 or CDK19 on CDK8 and CDK19 protein levels in HEK293 cells.

FIG. 7 shows that Senexin A inhibits NFκB activation with minimal effects on cell viability relative to proteasome-targeting NFκB inhibitors TPCK and MG115.

FIG. 8 shows that Senexin A does not block nuclear NFκB protein DNA binding, in contrast to proteasome-targeting NFκB inhibitors TPCK and MG115.

FIGS. 9A-9B show a variety of SNX2-class compounds useful in the methods according to the invention. FIG. 9A shows Cmpd Nos. 1-19. FIG. 9B shows Cmpd Nos. 20-47.

### DETAILED DESCRIPTION OF THE PREFERRED EMBODIMENTS

The present inventors have discovered compounds (called SNX2-class compounds) that selectively inhibit CDK8/19 and that not only inhibit the induction of NFκB transcriptional activity by p21 but, surprisingly, also prevent the induction of this activity by a canonical NFκB inducer TNF-α, which acts through a well-characterized mechanism unrelated to the CKI pathway. This discovery indicates that SNX2-class compounds and CDK8/19 inhibitors in general have utility in the treatment of a variety of diseases, including but not limited to inflammatory diseases, which are known to be caused by NFκB.

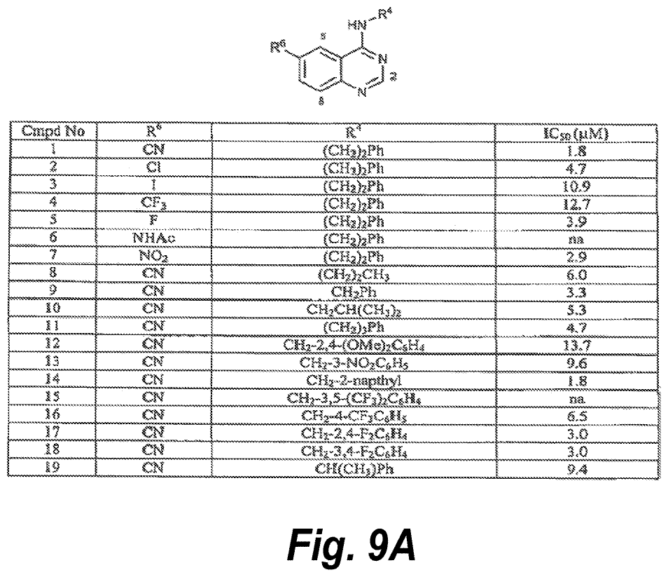

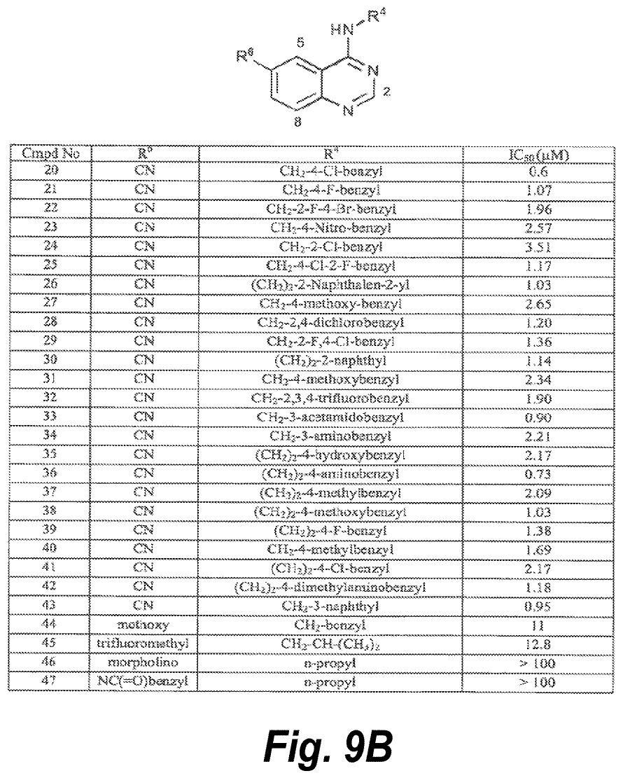

The invention provides a method for treating a disease or disorder in a mammal which is caused by induced NFκB transcriptional activity in cells of the mammal, the method comprising administering to the mammal a compound that specifically inhibits one or more of CDK8 and CDK19. In some embodiments, the induced NFκB transcriptional activity is not induced by the CKI pathway. In some embodiments, the NFκB transcriptional activity is induced via the canonical pathway, which in some embodiments may be by TNF-α, or by other canonical inducers. In some embodiments the induced NFκB transcriptional activity is inhibited without inhibiting the basal NFκB transcriptional activity. In some embodiments, the disease is an inflammatory disease. In some embodiments, the inflammatory disease is selected from the group consisting of asthma, inflammatory bowel disease and rheumatoid arthritis. In some embodiments, the inflammatory bowel disease is Chron's disease or ulcerative colitis. In some embodiments, the compound has a structure selected from the group of structures shown in FIGS. 9A-9B.

In embodiments where the induced transcriptional activity of NFκB is not induced by the CKI pathway, including embodiments where the induced transcriptional activity of NFκB is induced by the canonical pathway, the compound may have the structure

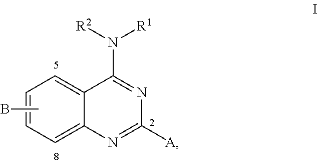

wherein

R¹ is selected from lower alkyl, aralkyl, aryl, heteroaryl, phenethyl, and alkoxyphenyl, any of which may be substituted or unsubstituted;

R² is selected from lower alkyl and hydrogen;

A is selected from hydrogen or lower alkyl; and

B is selected from halogen, cyano, trifluoromethyl, NHAc, NO₂, and O-lower alkyl.

In some embodiments, R¹ is selected from lower alkyl and aralkyl, which may be substituted or unsubstituted. In some embodiments, R¹ is aralkyl which may be unsubstituted, or monosubstituted or disubstituted with one or more of lower alkyl, O-lower alkyl, NO₂, halogen, acetamido and amino. In some embodiments, R¹ is aralkyl, wherein aryl is naphthyl.

The embodiments wherein the transcriptional activity of NFκB is not induced by the CKI pathway, including embodiments where the induced transcriptional activity of NFκB is induced by the canonical pathway, include methods for treating a disease caused by induced transcriptional activity. These embodiments also include methods for inhibiting induced transcriptional activity of NFκB, but not basal activity of NFκB in a mammalian cell. In some such embodiments, the mammalian cell is in the body of a mammal.

The term “disease or disorder” is intended to mean a medical condition associated with specific symptoms or signs. The term “caused by induced NFκB transcriptional activity in cells of the mammal” means that at least some of the symptoms or signs of the disease or disorder would not be present, but for the fact that at least some cells in the mammal have induced NFκB transcriptional activity. The term “induced NFκB transcriptional activity” means that the transcriptional function performed by NFκB is performed at greater than basal NFκB transcriptional activity level. The term “basal NFκB transcriptional activity” means the level of transcriptional function performed by NFκB in a cell under normal conditions, i.e., in the absence of the disease or disorder. In some embodiments, the amount of active NFκB in the nucleus of the cells is not increased, but rather only the level of NFκB activity is increased. The term “treating” means reducing or eliminating at least some of the signs or symptoms of the disease. The term “mammal” includes a human. The terms “administering”, “administration” and the like are further discussed below. The term “compound that specifically inhibits one or more of CDK8 and CDK19” means a small molecule that inhibits the activity of CDK8 and/or CDK19 to a greater extent than it inhibits the activity of one or more of CDK1, CDK2 and CDK6.

In some embodiments, a compound according to the invention is administered as a pharmaceutical formulation including a physiologically acceptable carrier. The term “physiologically acceptable” generally refers to a material that does not interfere with the effectiveness of the compound and that is compatible with the health of the mammal. The term “carrier” encompasses any excipient, diluent, filler, salt, buffer, stabilizer, solubilizer, oil, lipid, lipid containing vesicle, microspheres, liposomal encapsulation, or other material well known in the art for use in physiologically acceptable formulations. It will be understood that the characteristics of the carrier, excipient, or diluent will depend on the route of administration for a particular application. The preparation of physiologically acceptable formulations containing these materials is described in, e.g., Remington's Pharmaceutical Sciences, 18th Edition, ed. A. Gennaro, Mack Publishing Co., Easton, Pa., 1990. The active compound is included in the physiologically acceptable carrier or diluent in an amount sufficient to deliver to a patient a prophylactically or therapeutically effective amount without causing serious toxic effects in the patient treated. The term an “effective amount” or a “sufficient amount” generally refers to an amount sufficient to affect a reduction or elimination of at least one symptom or sign of the disease or disorder.

In the methods according to the invention, administration of a compound according to the invention can be by any suitable route, including, without limitation, parenteral, oral, intratumoral, sublingual, transdermal, topical, intranasal, aerosol, intraocular, intratracheal, intrarectal, mucosal, vaginal, by dermal patch or in eye drop or mouthwash form. Administration of the compound or pharmaceutical formulation can be carried out using known procedures at dosages and for periods of time effective to reduce symptoms or surrogate markers of the disease.

As described in the co-owned US patent publications 20080033000 and 20060154287, the instant inventors have conducted high-throughput screening (HTS) for CM pathway inhibition using diversified libraries comprising >100,000 drug-like small molecules. The screening assay uses a human HT1080-based reporter cell line that expresses p21 from an artificial isopropyl-β-thio-galactoside (IPTG)-inducible promoter and contains a p21-responsive cytomegalovirus (CMV) promoter driving GFP expression (Roninson and Chang, 2006). Among a small number of compounds identified by HTS, we have concentrated on a group of non-cytotoxic 4-aminoquinazolines, designated SNX2-class compounds (Chang et al., 2008). While SNX2-class compounds inhibit the induction of transcription by p21 and other CKIs, they do not interfere with CKI-induced cell cycle arrest (Chang et al., 2008). After identifying the original best hits (SNX2 and SNX14) (Chang et al., 2008), we have carried out lead optimization of SNX2-class compounds through de novo synthesis and structure-activity relationship (SAR) analysis, generating novel structures with up to 30-fold increase in potency in the CMV-based reporter assay (U.S. application Ser. No. 12/956,420). We have also determined that the optimized SNX2-class compounds selectively target two closely related kinases of the CDK family, CDK8 and CDK19, which function in the regulation of transcription rather than cell cycle progression (Malumbres et al., 2009). shRNA knockdown studies by instant inventors revealed that CDK8 but not CDK19 is the target of SNX2-class compounds, responsible for their activity as CKI pathway inhibitors in HT1080 cells (U.S. application Ser. No. 12/956,420).

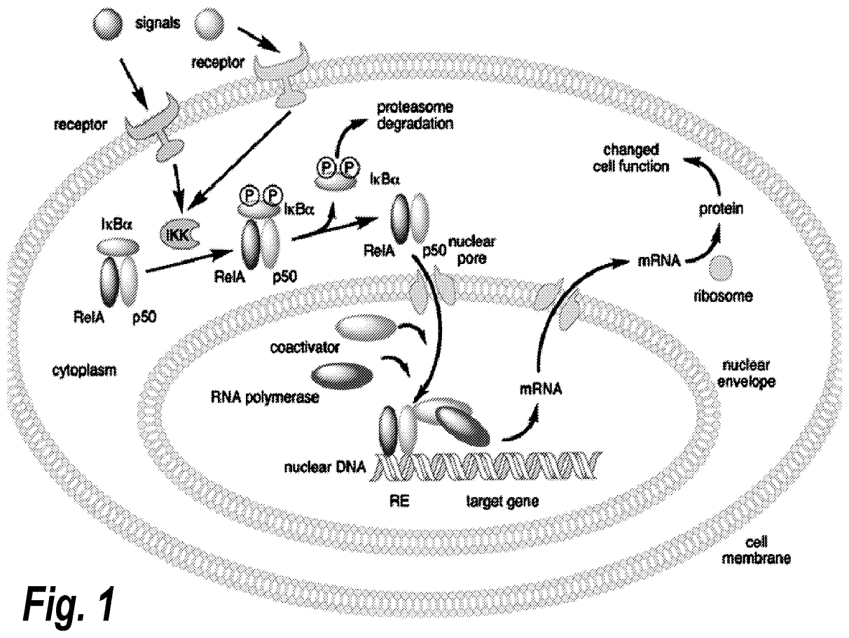

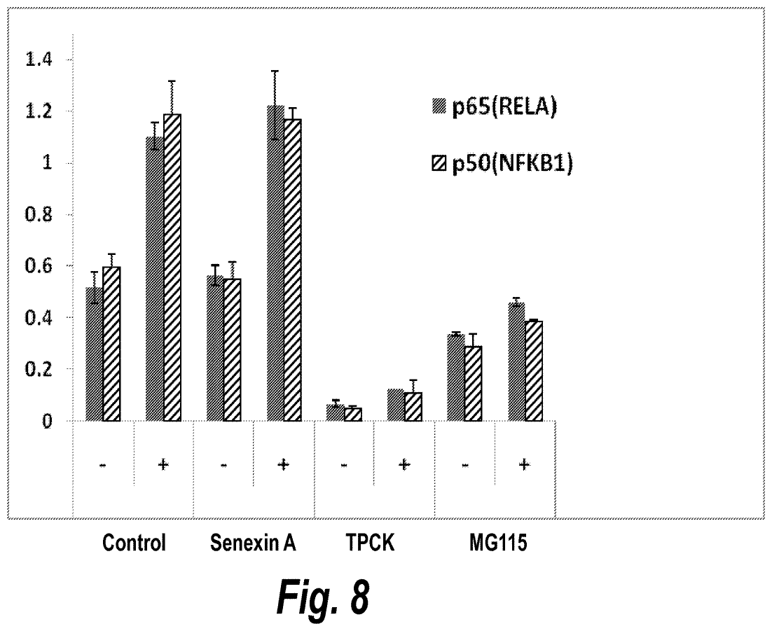

Given the role of NFxB in the induction of transcription by p21 (Poole et al., 2004), we have tested SNX2 for the ability to decrease the amount of active NFκB in the nucleus, a general assay for different known classes of NFκB inhibitors. As shown in FIG. 8 of US patent publication 20080033000, we have found, using ACTIVE MOTIF TransAM™ NFκB p65 Chemi and NFκB p50 Chemi Transcription Factor Assay Kits, that SNX2 had no effect on the amount of p50 or p65 NFκB subunits binding to NFκB consensus sequence in nuclear extracts from HT 1080 cells, untreated or treated with NFκB inducer TNFα. This lack of effect suggested at the time that SNX2-class compounds do not act via NFκB inhibition. As described in Example 1 below, however, we have now discovered that these compounds not only inhibit the induction of NFκB transcriptional activity by p21 but, surprisingly, also prevent the induction of this activity by a canonical NFκB inducer TNFα, which acts through a well-characterized mechanism (FIG. 1) unrelated to the CKI pathway. This discovery indicates that SNX2-class compounds and CDK8/19 inhibitors in general have utility in the treatment of a variety of diseases, including but not limited to inflammatory diseases, which are known to be mediated by NFκB.

As previously demonstrated in US patent publication 20080033000, SNX2-class CKI pathway inhibitors have utility in various diseases associated with the CM pathway, such as cancer, viral diseases, Alzheimer's disease, and atherosclerosis. The utility of CKI pathway inhibitors was expected to be inherently limited to the responses that are mediated by p21 or other CKI proteins. The present invention demonstrates that SNX2-class compounds inhibit the induction of NFκB by TNFα, a signal that activates NFκB through the canonical pathway (FIG. 1), in which p21 or other CKI proteins have not been implicated. This discovery demonstrates that SNX2-class compounds should be useful in the treatment of any diseases that involve NFκB activation, regardless of CKI protein involvement. Since SNX2-class compounds are selective CDK8/19 inhibitors, any other CDK8/19 inhibitors are expected to have the same activity.

Although numerous NFκB inhibitors are known, SNX2-class compounds appear to have a unique combination of properties which is not known to be shared by any other NFκB inhibitors and that bodes well for the utility of SNX2-class compounds in chronic diseases. SNX2-class compounds are not cytotoxic. They inhibit NFκB transcriptional activity induced by TNFα or by a stress-response protein p21, and they do not inhibit the basal NFκB activity, suggesting that these compounds may not have toxicity that could result from NFκB inhibition under normal conditions. Furthermore, SNX2-class compounds inhibit NFκB induction through a different mechanism than the known inhibitors, as indicated by the inability of SNX2-class compounds to decrease basal or TNFα-induced amounts of active NFκB in the nucleus. This lack of activity is incompatible with the inhibition of those steps in the NFκB pathway that are commonly targeted by known NFκB inhibitors (FIG. 1) but it is compatible with those steps where SNX2-class compounds are likely to act based on the nature of their selection (against the effect of p21) and their molecular target (CDK8/19). Specifically, p21 stimulates the coactivating effect of p300/CBP on NFκB (Vazquez et al., 2005; Snowden et al., 2000; Gregory et al., 2002; Garcia-Wilson and Perkins, 2005), a potential target step for SNX2-class compounds. In addition, CDK8 and CDK19 are involved in Pol II interaction with transcription factors (Sato et al., 2004), suggesting that inhibition of this interaction may mediate the effect of SNX2-class compounds on NFκB (FIG. 1). An effect on either p300/CBP or Pol II (neither of which are targeted by known NFκB inhibitors) would be expected to influence the transcriptional activity but not the amount of active NFκB in the nucleus, as observed for SNX2-class compounds.

The list of known NFκB inhibitors includes pan-tropic CDK inhibitors, flavopiridol and R-roscovitine (Gupta et al., 2010). However, the effects of these compounds on NFκB were reported to be due to IKK inhibition (Takada and Aggarwal, 2004; Dey et al., 2008), a mechanism which is incompatible with the inability of SNX2-class compounds to block the increase in the nuclear content of active NFκB (Chang et al., 2008). Pan-tropic CDK inhibitors have a broad antiproliferative activity and have shown pronounced toxicity in clinical trials (Diaz-Padilla et al., 2009). In contrast, SNX2-class compounds have no antiproliferative activity at their active concentrations. Furthermore, CDK8 knockdown or knockout did not inhibit cell growth (Westerling et al., 2007), suggesting that the role of CDK8 could be limited to early embryonic development, and that CDK8 inhibitors could be safe for prolonged treatment outside of pregnancy. These considerations suggest that SNX2-class compounds, the first selective inhibitors of CDK8/19, may be safer for long-term administration than other CDK inhibitors or NFκB inhibitors, and may therefore be suitable for therapeutic applications in chronic diseases, in particular inflammatory diseases, including inflammatory arthritis.

The following examples are intended to further illustrate the invention and are not to be construed to limit the scope of the invention.

### Example 1

### SNX2-Class Compounds Inhibit the Induction of NFκB Transcriptional Activity

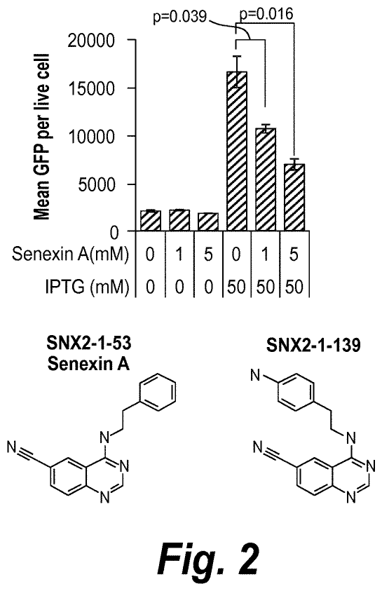

We have tested the effects of SNX2-class compounds on NFκB transcriptional activity. These assays were conducted with a reporter cell line that we derived from HT1080 p21-9 cells carrying IPTG-inducible p21 (Chang et al., 1999) after transduction with Cignal Lenti NFκB Reporter lentivirus (SA Biosciences), which expresses GFP from a NFκB-dependent minimal promoter. The reporter cell line was then selected for a high basal level of NFκB-dependent GFP expression, which was further increased by TNFα or upon p21 induction by IPTG. SNX2-class compounds strongly inhibited the induction of the NFκB-dependent promoter by p21, as illustrated for SNX2-1-53 (a.k.a. Senexin A) by a flow cytometric experiment in FIG. 2, where cells were untreated or treated with 50 mM of p21-inducing IPTG for 72 hrs, in the absence or in the presence of different concentrations of Senexin A.

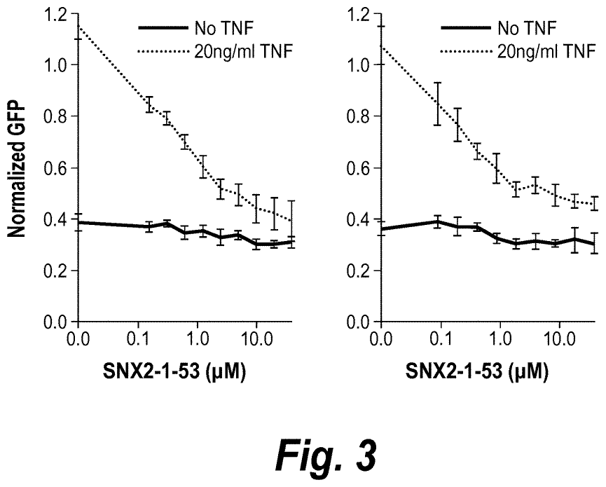

The ability of SNX2-class compounds to prevent the induction of the NFκB-dependent promoter by p21 was not surprising, since these compounds were identified by their ability to prevent p21-mediated induction of another promoter (CMV) (Chang et al., 2008), and NFκB stimulation by p21 was already known. Unexpectedly, however, we found that SNX2-class compounds also inhibited the induction of the NFκB-dependent promoter by a canonical NFκB inducer TNFα, as illustrated in FIG. 3 for two SNX2-class compounds, SNX2-1-53 and SNX2-1-139 (the structures of these compounds are shown in FIG. 2). The same HT1080-based NFκB-GFP reporter cell line, untreated or treated with 10 ng/ml TNFα for 18 hrs, in the absence or in the presence of different concentrations of SNX2-class compounds, was analyzed in a 96-well fluorometric assay, where GFP expression was normalized by Hoechst 33342 DNA staining Both SNX2-class compounds inhibited TNFα-induced NFκB activity, reaching a plateau of inhibition at the level approximating that of untreated cells, but they did not significantly inhibit the basal NFκB activity.

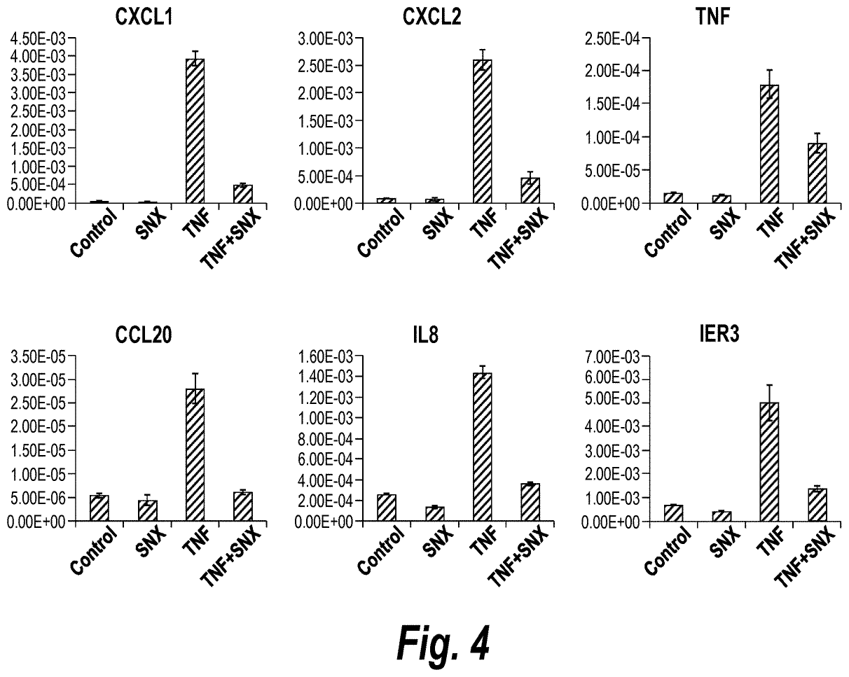

The effect of Senexin A on TNFα-induced transcription was also demonstrated in human renal HEK293 cells (FIG. 4). The cells were seeded in 6-well plates at 6×10⁵ cells/well in media containing 3% serum and cultured overnight. The next day, cells were pretreated with 5 μM Senexin A or with DMSO vehicle control for 1 hour and treated with or without 10 ng/ml TNFα for 30 minutes. Cells were then lysed for total RNA purification with the RNeasy Kit (Qiagen). For QPCR analysis of NFκB-inducible genes, cDNA was prepared using Maxima First Strand cDNA Synthesis Kit (Thermo Scientific/Fermentas, K1641) and gene expression was measured by QPCR with gene-specific primers, with RPL13A as a normalization standard, using Maxima SYBR Green/ROX qPCR Master Mix (Thermo Scientific/Fermentas, K0223) and ABI Prism 7900HT Detection system (Life technologies). The primer sequences used for QPCR are listed in Table I.

TABLE 1
PRIMER SEQUENCES FOR QPCR.

| | Sense | Antisense |
|---|---|---|
| Gene | (SEQ ID NO) | (SEQ ID NO) |
| RPL13A | GGCCCAGCAGTACCTGTTTA (1) | AGATGGCGGAGGTGCAG (2) |
| IL8 | AAATTTGGGGTGGAAAGGTT (3) | TCCTGATTTCTGCAGCTCTGT (4) |
| CXCL1 | AACAGCCACCAGTGAGCTTC (5) | GAAAGCTTGCCTCAATCCTG (6) |
| IER3 | ACACCCTCTTCAGCCATCAG (7) | CGCAGGGTTCTCTACCCTC (8) |
| CXCL2 | GCTTCCTCCTTCCTTCTGGT (9) | GGGCAGAAAGCTTGTCTCAA (10) |
| CCL20 | GGGCAGAAAGCTTGTCTCAA (11) | GTGCTGCTACTCCACCTCTG (12) |
| TNF | TCAGCCTCTTCTCCTTCCTG (13) | GCCAGAGGGCTGATTAGAGA (14) |
| ERG1 | AGCCCTACGAGCACCTGAC (15) | AAAGCGGCCAGTATAGGTGA (16) |

All the tested genes were induced by TNFα but Senexin A treatment drastically inhibited such induction (FIG. 4).

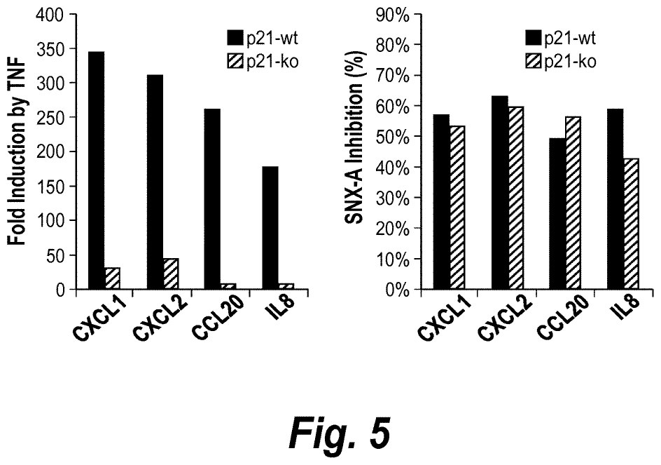

We have verified the effect of CDK8/19 inhibition on NFκB-mediated induction of transcription in human HCT116 colon carcinoma cells, where we also used the availability of a p21−/− derivative of this cell line (Waldman et al., 1996) to determine if this effect depends on p21. The wild-type and p21−/− HCT116 cells were seeded in 6-well plates at 6×10⁵ cells/well in media with 10% serum and cultured overnight. The next day, cells were pretreated with 5 μM Senexin A or with DMSO vehicle control for 1 hour and treated with or without 10 ng/ml TNFα for 30 minutes. Cells were then lysed for RNA purification and QPCR analysis of NFκB-inducible genes. FIG. 5 (left panel) shows fold induction of the indicated genes by TNFα treatment, in the absence of Senexin A. All the genes were induced by TNFα in both cell lines, but their fold induction was much diminished by p21 knockout. FIG. 5 (right panel) shows the inhibitory effects of Senexin A treatment on TNFα-induced gene expression in both cell lines. Remarkably, Senexin A inhibited TNFα-induced gene expression to the same degree in the wild-type and p21−/− cells, demonstrating that the effect of CDK8/19 inhibition on NFκB-mediated induction of transcription is independent of p21.

### Example 2

### Both CDK8 and CDK19 Play a Role in NFκB Activation

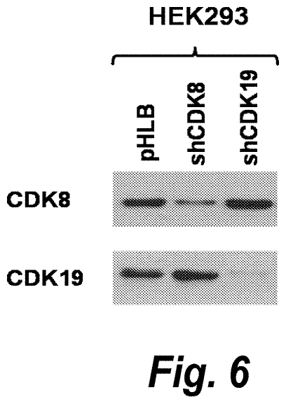

To verify that CDK8 and/or CDK19 mediate NFκB-induced transcription, we have used shRNAs targeting CDK8 and CDK19 to knock down the expression of these genes in HEK293 cells. HEK293 cells were transduced with pHLB-based lentiviral vectors, derived from pLKO.1 lentiviral vector and carrying the blasticidin resistance marker, and expressing shRNAs against CDK8 (targeted sequence CCTCTGGCATATAATCAAGTT (SEQ ID NO: 17)) or CDK19 (targeted sequence GCTTGTAGAGAGATTGCACTT (SEQ ID NO: 18)). After blasticidin selection of lentivirus-infected cells, the knockdown of CDK8 and CDK19 were confirmed at the protein level by immunoblotting, as shown in FIG. 6. The following primary antibodies were used for immunoblotting: goat-anti-CDK8 (Santa Cruz, sc-1521), rabbit-anti-CDK19 (Sigma, HPA007053). To test the effects of CDK8 and CDK19 knockdown on the induction of NFκB-regulated genes by TNFα, control (pHLB-transduced) and CDK8 or CDK19 knockdown cells were seeded in 6-well plates at 6×10⁵ cells/well in media with 10% serum and cultured overnight before treatment with or without 10 ng/ml TNFα for 30 minutes. Total RNA was purified and gene expression was measured by QPCR. The results of this analysis are shown in Table II.

TABLE II
Fold induction of the indicated genes by TNFα.

| — | CCL20 | CXCL1 | EGR1 | IL8 | TNF |
|---|---|---|---|---|---|
| pHLB | 6.61 | 106.16 | 2.29 | 6.50 | 8.67 |
| shCDK8 | 4.11 | 49.68 | 1.54 | 4.47 | 8.98 |
| shCDK19 | 3.33 | 56.57 | 1.17 | 3.90 | 4.32 |

These results demonstrate that both CDK8 and CDK19 are positive mediators of the induction of NFκB-mediated transcription, and therefore compounds that inhibit both CDK8 and CDK19 (such as SNX2-class compounds) are the most advantageous for this effect

### Example 3

### CDK8/19 Inhibitor Inhibits NFκB Through a Different Mechanism than Other NFκB Inhibitors

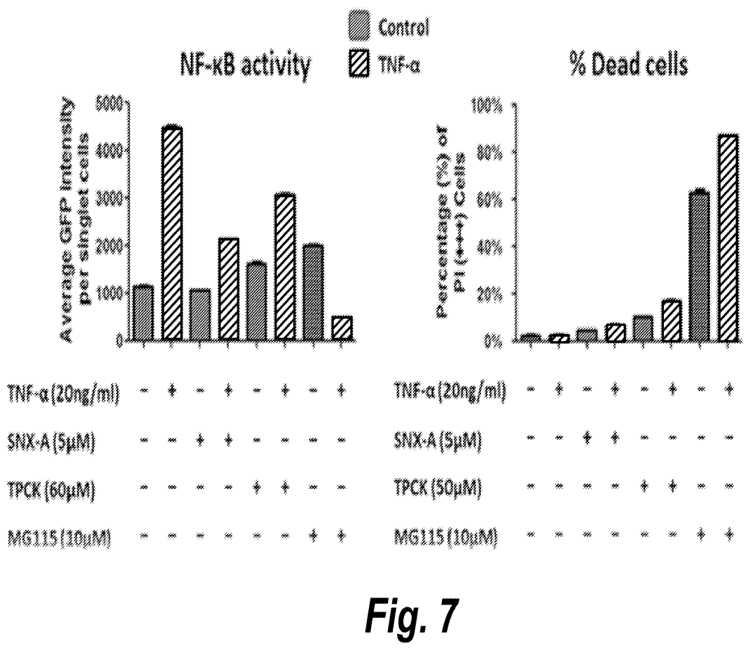

We have compared Senexin A to two known proteasome-targeting NFκB inhibitors, N-tosyl-L-phenylalanine chloromethyl ketone (TPCK) (Ha et al., 2009) and MG115 in regard to their cytotoxicity and their effect on the nuclear translocation of active NFκB. In the experiment shown in FIG. 7, the HT1080-derived NFkB-GFP reporter cell line was seeded in 60 mm plates at 1.5×10⁵ cells per plate and cultured overnight before being treated with different NFκB inhibitors at the concentrations indicated in FIG. 7 for 3 hours, followed by 18 hours TNFα (10 ng/ml) stimulation. The treated cells were trypsinized, resuspended in PBS, mixed with 5 μg/ml propidium iodide (PI), and analyzed using LSRII flow cytometer (BD Biosciences) for GFP fluorescence (left panel) and the percentage of dead (PI-positive) cells (right panel). Senexin A, TPCK and MG115 all inhibited TNFα-induced NFκB-dependent transcription, but TPCK and MG115 strongly increased the fraction of dead cells, whereas Senexin A did not.

The DNA-binding activities of nuclear NFκB proteins were measured by the ELISA-based TransAM NFκB Family Transcriptional Factor Assay Kit (Active Motif) following manufacturer's protocol. HT1080 and HEK293 cells were pretreated with inhibitors (5 μM Senexin A, 60 μM TPCK, 10 μM MG115) for 3 hours and then treated with 10 ng/ml TNF for 30 minutes before nuclear extract preparation with Nuclear Extraction Kit (Active Motif). Nuclear extracts were assayed at 5 μg/well for p65 and 2.5 μg/well for p50 DNA binding. FIG. 8 shows the results from assays conducted in duplicate. TPCK and MG115 strongly decreased the amount of active p65 and p50 in the untreated and TNFα-treated cells of both cell lines. In contrast, the results with Senexin A were indistinguishable from the control, indicating that the CDK8/19 inhibitor does not inhibit nuclear translocation of NFκB. In agreement with this finding, we have previously reported that SNX2, a compound related to Senexin A, also fails to inhibit the nuclear levels of active NFκB (Chang et al., 2008).

Hence, CDK8/19 inhibitors inhibit NFκB through a novel combination of properties: (i) they inhibit the TNFα-induced but not the basal NFκB transcriptional activity, (ii) they are not cytotoxic, and (iii) they do not inhibit the nuclear translocation of active NFκB. This unique combination of properties can be explained by the likely mechanisms of action of SNX2-class CDK8/19 inhibitors (FIG. 1): CDK8/19 could act on p300/CBP coactivators, which are stimulated by p21 (Vazquez et al., 2005; Snowden et al., 2000), or on NFκB interaction with Pol II, which is regulated by CDK8/19-containing Mediator complexes (Sato et al., 2004).

The references cited herein are hereby incorporated by reference in their entirety. Any discrepancy between the teachings of any cited reference and the teachings of this specification shall be resolved in favor of the latter.

Those skilled in the art will recognize that equivalents of the claimed invention will exist and are covered by the claims.

1.-19. (canceled)

20. A method for treating an inflammatory disease or disorder in a mammal which is caused by induced NF-κB transcriptional activity in cells of the mammal, the method comprising administering to the mammal a compound that specifically inhibits one or more of CDK8 and CDK19, wherein the induced NF-κB transcriptional activity is inhibited without inhibiting basal NF-κB transcriptional activity.

21. The method of claim 20, wherein the NF-κB transcriptional activity has not been induced via the CKI pathway.

22. The method of claim 20, wherein the NF-κB transcriptional activity has been induced via the canonical pathway.

23. The method of claim 22, wherein the NF-κB transcriptional activity has been induced by TNF-α.

24. The method of claim 20, wherein the compound has a structure selected from the group of structures shown in FIG. 9A or FIG. 9B.

25. The method of claim 20, wherein the compound has the structure 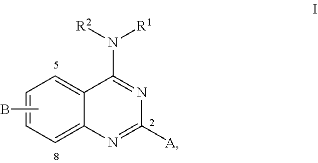
wherein
R¹ is aralkyl, wherein aryl is naphthyl;
R² is selects from lower alkyl and hydrogen;
A is selected from lower alkyl and hydrogen; and
B is selected from halogen, cyano, trifluoromethyl, NHAc, NO₂, and O-lower alkyl.

26. The method of claim 25, wherein B is cyano.

27. The method of claim 26, wherein R² is hydrogen and A is hydrogen.

28. The method of claim 25, wherein the compound has the structure 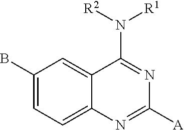
wherein
R¹ is selected from lower alkyl, aralkyl, aryl, heteroaryl, phenethyl, and alkoyxphenyl, any of which may be substituted or unsubstituted;
R² is selectes from lower alkyl and hydrogen;
A is selected from lower alkyl and hydrogen; and
B is cyano.

29. The method of claim 28, wherein R¹ is aralkyl, wherein aryl is naphthyl.

30. The method of claim 29, wherein R² is hydrogen and A is hydrogen.

31. The method of claim 20, wherein the inflammatory disease or disorder is selected from the group consisting of asthma, inflammatory bowel disease, and rheumatoid arthritis.

32. A method for inhibiting induced NF-κB transcriptional activity in a mammalian cell, wherein the NF-κB transcriptional activity is not induced via the CKI pathway, the method comprising contacting the cell with a compound having the structure 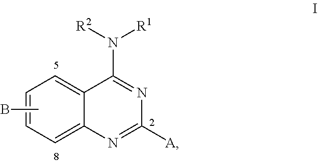
wherein
R¹ is aralkyl, wherein aryl is naphthyl;
R² is selects from lower alkyl and hydrogen;
A is selected from lower alkyl and hydrogen; and
B is selected from halogen, cyano, trifluoromethyl, NHAc, NO₂, and O-lower alkyl.

33. The method of claim 32, wherein B is cyano.

34. The method of claim 33, wherein R² is hydrogen and A is hydrogen.

35. The method of claim 32, wherein the compound has the structure 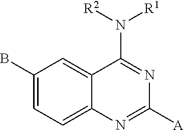
wherein
R¹ is selected from lower alkyl, aralkyl, aryl, heteroaryl, phenethyl, and alkoyxphenyl, any of which may be substituted or unsubstituted;
R² is selectes from lower alkyl and hydrogen;
A is selected from lower alkyl and hydrogen; and
B is cyano.

36. The method of claim 35, R¹ is aralkyl, wherein aryl is naphthyl.

37. The method of claim 36, wherein R² is hydrogen and A is hydrogen.

38. The method of claim 32, wherein the NF-κB transcriptional activity is induced via the canonical pathway.

39. The method of claim 32, wherein the mammalian cell is in the body of a mammal.


SEQUENCE LISTING (18 sequences)

SEQ ID NO: 1 - DNA, 20 bp, Artificial Sequence
  Synthetic primer for gene RPL13A
ggcccagcagtacctgttta

SEQ ID NO: 2 - DNA, 17 bp, Artificial Sequence
  Synthetic primer for gene RPL13A
agatggcggaggtgcag

SEQ ID NO: 3 - DNA, 20 bp, Artificial Sequence
  Synthetic primer for gene IL8
aaatttggggtggaaaggtt

SEQ ID NO: 4 - DNA, 21 bp, Artificial Sequence
  Synthetic primer for gene IL8
tcctgatttctgcagctctgt

SEQ ID NO: 5 - DNA, 20 bp, Artificial Sequence
  Synthetic primer for gene CXCL1
aacagccaccagtgagcttc

SEQ ID NO: 6 - DNA, 20 bp, Artificial Sequence
  Synthetic primer for gene CXCL1
gaaagcttgcctcaatcctg

SEQ ID NO: 7 - DNA, 20 bp, Artificial Sequence
  Synthetic primer for gene IER3
acaccctcttcagccatcag

SEQ ID NO: 8 - DNA, 19 bp, Artificial Sequence
  Synthetic primer for gene IER3
cgcagggttctctaccctc

SEQ ID NO: 9 - DNA, 20 bp, Artificial Sequence
  Synthetic primer for gene CXCL2
gcttcctccttccttctggt

SEQ ID NO: 10 - DNA, 20 bp, Artificial Sequence
  Synthetic primer for gene CXCL2
gggcagaaagcttgtctcaa

SEQ ID NO: 11 - DNA, 20 bp, Artificial Sequence
  Synthetic primer for gene CCL20
cgtgtgaagcccacaataaa

SEQ ID NO: 12 - DNA, 20 bp, Artificial Sequence
  Synthetic primer for gene CCL20
gtgctgctactccacctctg

SEQ ID NO: 13 - DNA, 20 bp, Artificial Sequence
  Synthetic primer for gene TNF
tcagcctcttctccttcctg

SEQ ID NO: 14 - DNA, 20 bp, Artificial Sequence
  Synthetic primer for gene TNF
gccagagggctgattagaga

SEQ ID NO: 15 - DNA, 19 bp, Artificial Sequence
  Synthetic primer for gene EGR1
agccctacgagcacctgac

SEQ ID NO: 16 - DNA, 20 bp, Artificial Sequence
  Synthetic primer for gene EGR1
aaagcggccagtataggtga

SEQ ID NO: 17 - DNA, 21 bp, Artificial Sequence
  Synthetic oligonucleotide
cctctggcatataatcaagtt

SEQ ID NO: 18 - DNA, 21 bp, Artificial Sequence
  Synthetic oligonucleotide
gcttgtagagagattgcactt